(19) United States
(12) Patent Application Publication
(10) Pub. No.: US 2023/0390350 A1
(43) Pub. Date: Dec. 7, 2023

(54) TREATMENT OF ORAL MUCOSITIS

(71) Applicant: IZUN PHARMACEUTICALS CORP., New York, NY (US)

(72) Inventors: Shmuel HESS, Ness Ziona (IL); William Z. LEVINE, Jerusalem (IL)

(73) Assignee: IZUN PHARMACEUTICALS CORP., New York, NY (US)

(21) Appl. No.: 18/250,760

(22) PCT Filed: Oct. 27, 2021
(86) PCT No.: PCT/IL2021/051274

§ 371 Date: Apr. 27, 2023

Related U.S. Application Data
(60) Provisional application No. 63/106,364, filed on Oct. 28, 2020.

(51) Int. Cl. A61K 36/28; A61P 1/02; A61K 36/23; A61K 9/00

(52) U.S. Cl. CPC A61K 36/28; A61P 1/02; A61K 36/23; A61K 9/006; A61K 2236/333

(57) ABSTRACT

Described herein are methods for treatment of oral mucositis comprising administering to a patient in need thereof a composition comprising extracts from the plant species Sambucus nigra, Echinacea purpurea, and Centella asiatica; in patients treated with a once-weekly regimen of 30-40 mg/m² of cisplatin.

### FIELD

Provided herein are methods for treatment of oral mucositis.

### BACKGROUND

Oral mucositis (OM) is a common and painful side effect of drug and radiation therapy for cancer, in particular head and neck cancer. The disorder is characterized by inflammation within the oral mucosa that can lead to breakdown of the oral mucosa that results in the formation of ulcerative lesions.

OM is among the most frequently reported and potentially most debilitating conditions associated with cancer chemotherapy and radiation treatment, ranging in incidence from 10%-75% in patients receiving chemotherapy or radiotherapy, 70-90% in bone marrow transplant recipients and >95% of patients receiving combination radiation and chemotherapy for head and neck cancers (HNC). OM has been associated with increased analgesic and antibiotic use, febrile days, need for gastric tube or parenteral nutrition, length of hospital stay, unplanned and emergency room visits and total medical expenses, all of which have a negative impact on health and economic outcomes. Approximately 500,000 patients develop OM in the United States annually, and it is considered largely unpreventable.

Standard therapy for OM is predominantly palliative, including application of topical analgesics such as lidocaine, rinses and mouthwashes, and/or systemic administration of narcotics and antibiotics. Currently, the only approved treatment for oral mucositis is Kepivance (KGF-1, palifermin) and its use is confined to patients receiving conditioning regimens in preparation for stem cell transplants for the treatment of hematologic malignancies.

### SUMMARY

Described herein are methods for treatment of oral mucositis comprising administering to a patient in need thereof a composition comprising extracts from the plant species Sambucus nigra, Echinacea purpurea, and Centella asiatica.

The foregoing and other objects, features, and advantages will become more apparent from the following detailed description, which proceeds with reference to the accompanying FIGURES.

### DETAILED DESCRIPTION

### Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

### Overview of Several Embodiments

Provided herein are methods for treatment and/or prevention of oral mucositis in a patient undergoing a chemotherapeutic treatment, using a composition comprising at least 1% herbal extract comprising Sambucus nigra, Echinacea purpurea, and Centella, such as Centella asiatica. In an embodiment, the ratio of Sambucus nigra:Echinacea purpurea:Centella asiatica is 7:1:2. Optionally, the herbal extracts are extracts produced by a first hydroalcoholic extraction from plant matter, followed by a second hydroalcoholic extraction of the first extract. The hydroalcoholic extracts for the first hydroalcoholic extraction and/or the second hydroalcoholic extraction may be prepared using an alcohol/water solution comprising between 50% and 90% alcohol, preferably, 70% alcohol. Optionally, the first extracts are prepared by combining plant matter with a hydroalcoholic solvent at a weight ratio of 1:8. Preferably, the patient is undergoing a treatment comprising cisplatin. The patient may be further undergoing a radiation therapy, optionally at a cumulative dose of up to 65 Gray (Gy), optionally a dose of 50 Gy. Optionally, the patient is suffering from head and neck cancer. The patient, optionally, is undergoing a once weekly cisplatin treatment regimen, optionally at a dosage of 30-40 milligrams per square meter (mg/m²).

Optionally, the patient suffered from oral pain at baseline, before administering the herbal extract containing composition, or before starting the chemotherapeutic treatment. Optionally, the pain is associated with prior surgical therapy.

Optionally, the composition is in the form of an oral rinse, preferably containing 1% botanical extracts. Optionally, the composition is administered in an amount of 15 ml. Optionally, the patient rinses with the oral rinse 3 times per day. The oral rinse is used, preferably, daily throughout the chemotherapy treatment regimen. Other compositions comprising the botanical extracts may be a gel, a patch or a lotion.

Additionally provided herein are methods for treatment of pain associated with oral mucositis in a patient undergoing a chemotherapeutic treatment with cisplatin, comprising administering to the patient the compositions described above. Additionally provided herein are methods for treatment of pain associated with oral surgery in a patient undergoing a chemotherapeutic treatment with cisplatin, comprising administering to the patient the compositions described above.

Additionally provided herein are compositions for treatment of oral mucositis in patients treated with a once-weekly regimen of 30-40 mg/m² of cisplatin, comprising, extracts from the plant species Sambucus nigra, Echinacea purpurea, and Centella asiatica.

Additionally provided herein are compositions for treatment of pain associated with oral mucositis in patients treated with a once-weekly regimen of 30-40 mg/m² of cisplatin, comprising, extracts from the plant species Sambucus nigra, Echinacea purpurea, and Centella asiatica.

Additionally provided herein are compositions for treatment of pain associated with oral surgery in patients treated with a once-weekly regimen of 30-40 mg/m² of cisplatin, comprising, extracts from the plant species Sambucus nigra, Echinacea purpurea, and Centella asiatica.

The following examples are provided to illustrate certain particular features and/or embodiments. These examples should not be construed to limit the disclosure to the particular features or embodiments described.

### Examples

### Example 1: Preparation of Herbal Extract

Sambucus nigra (flowering tops) was mixed with 70% ethanol (8:1 solvent to plant ratio). Upon removing insoluble plant matter and drying solvent 3.29 kg of dried Sambucus nigra extract were formed.

Echinacea purpurea (rhizome and roots) was mixed with 70% ethanol (8:1 solvent to plant ratio). Upon removing insoluble plant matter and drying solvent 470 g (grams) of dried Echinacea purpurea extract were formed.

Centella asiatica (aerial parts) was mixed with 70% ethanol (8:1 solvent to plant ratio). Upon removing insoluble plant matter and drying solvent 940 g of dried Centella asiatica extract were formed.

The three dried extracts from the three herbs (ratio of 70:10:20 by weight) were combined, then 47 L of water were added and the mixture was stirred for 12 hours. 113.9 L of 96% ethanol were added to the mixture to form 160.9 L of a 70% ethanol alcoholic mixture, and stirred. The mixture was filtered and the insoluble material was removed. Ethanol was evaporated and the solution was spray-dried to form 3.2 kg of a dry extract powder. The yield of this step was 68.7%.

### Example 2: Preparation of Mouth Rinse

2.601 kg of dried extract prepared according to example 1 was stirred for 12 hours with kg of propylene glycol and 26.01 g sucralose to form a concentrate solution. 2.5 g of concentrate solution were mixed with 47.5 ml of saline solution to prepare a oral rinse. This oral rinse was designated as composition 1.

### Example 3: Study of Mouth Rinse in Oral Mucositis Patients

A Phase II study was performed, evaluating the effect of an oral rinse designated Composition 1 (as prepared in Example 2) for the prevention of chemoradiation-induced oral mucositis (OM) in patients with head and neck cancer. A multicenter, randomized, double-blind, placebo-controlled study was performed. Eligible subjects were randomized to receive either active drug or placebo for 5-9 weeks; treatment was concurrent with chemoradiotherapy (CRT) and extended until resolution of mucositis or up to 2 weeks after last CRT. Efficacy data included the OM WHO severity grades at each study visit and patient-reported Mouth and Throat Soreness (MTS) ratings collected from daily diaries.

Rinse dose is 15 ml of 1% oral rinse of composition 1, as described in example 2, at a frequency of three times daily. The placebo is prepared using Propylene Glycol, sucralose and food coloring, diluted in saline.

Subjects were scheduled to receive a continuous course of external beam irradiation delivered either through intensity modulated radiotherapy or 3D planning. The cumulative prescription dose is between 50-70 Gy. A minimum of 25% of the oral cavity receives a dose of 50 Gy or more. Radiotherapy is delivered concurrently with cisplatin chemotherapy in a dose of either 60-100 mg/m² (Q3W group), administered once every 21 days, or 30-40 mg/m², (Q1W group) administered once a week.

Safety was evaluated by general toxicity based on vital signs and physical examinations. Efficacy is evaluated by proportion of patients in active treatment group versus placebo group scoring a 3-4 according to WHO (World Health Organization) oral toxicity scale for OM at a cumulative radiation dosage of 50 Gy.

WHO oral toxicity scale for OM was evaluated as follows: Grade 0: No mucositis or mucosal lesions. Grade 1: erythema, mucosal sensitivity and pain. Grade 2: Ulceration, ability to eat solid foods. Grade 3: Ulceration, oral intake limited to fluids. Grade 4: Ulceration, oral feeding is impossible.

The following populations were used for analysis:
Intent-to-Treat (ITT): ITT includes all randomized subjects with baseline and post-baseline efficacy data.
Per Protocol Set (PPS): The PPS consists of all ITT subjects who reached a cumulative radiation dose of 50 Gy or dropped out with Grade 3 or 4 OM severity grades.
   The disposition of patients in the study is summarized in the table below:

TABLE 1
Disposition

| | | Treatment Group | | | | | |
|---|---|---|---|---|---|---|---|
| | | Compo- | | | | | |
| Number of Subjects | | sition 1 | | Placebo | | Total | |
| Randomized | | 64 | | 52 | | 116 | |
| Discontinued | | 29 | (45.3%) | 22 | (42.3%) | 51 | (44.0%) |
| Reasons for | Adverse events | 6 | (9.4%) | 3 | (2.4%) | 9 | (7.8%) |
| discon- | Withdrew | 10 | (15.6%) | 14 | (26.9%) | 24 | (20.7%) |
| tinuation | consent | | | | | | |
| | Lost to follow- | 6 | (9.4%) | 4 | (7.7%) | 10 | (8.6%) |
| | up | | | | | | |
| | Other reasons | 4 | (6.3%) | 1 | (1.9%) | 5 | (4.3%) |
| | Prohibited | 1 | (1.6%) | 0 | | 1 | (0.9%) |
| | meds | | | | | | |
| | Protocol | 2 | (3.1%) | 0 | | 2 | (1.7%) |
| | violation | | | | | | |

| | | | | | | |
|---|---|---|---|---|---|---|
| Completed | 35 | (54.5%) | 30 | (57.7%) | 65 | (56.0%) |

Overall, 116 patients were randomized to receive active treatment or placebo, and 44% discontinued the study, primarily due to withdrawn consent. The adverse event rate was relatively low and only slightly higher in the active treatment group versus placebo. To investigate the drop-out pattern further, Tables 2 and 3 summarize the number (%) of patients evaluated for OM at each study visit for the ITT and Per Protocol populations. In general, compliance with the OM assessment schedule was similar in the two groups, i.e., there was no clear difference in the pattern of drop-outs over time. Treatments commenced on visit 2, which is the baseline, when radiation therapy was initiated. Radiation therapy was performed daily and OM measurements were performed twice a week.

TABLE 2A
Duration of Follow-Up (ITT)

| | | Number of Subjects with OM Assessments | | | | | | | | | |
|---|---|---|---|---|---|---|---|---|---|---|---|
| Treatment Group | | V2 | V2 mid | V3 | V3 mid | V4 | V4 mid | V5 | V5 mid | V6 | V6 mid |
| Composition | n | 56 | 50 | 52 | 50 | 52 | 46 | 48 | 39 | 47 | 37 |
| 1 | % | 98 | 88 | 91 | 88 | 91 | 81 | 84 | 68 | 82 | 65 |
| Placebo | n | 50 | 43 | 49 | 45 | 46 | 41 | 42 | 39 | 41 | 38 |
| | % | 96 | 83 | 94 | 87 | 88 | 79 | 81 | 75 | 79 | 73 |

TABLE 2B
Duration of Follow-UP (ITT)

| | | Number of Subjects with OM Assessments | | | | | | | |
|---|---|---|---|---|---|---|---|---|---|
| | | | V7 | | V8 | | | | |
| Treatment Group | | V7 | mid | V8 | mid | V9 | V10 | V11 | V12 |
| Composition 1 | n | 41 | 38 | 40 | 19 | 21 | 17 | 47 | 28 |
| | % | 72 | 67 | 70 | 33 | 37 | 30 | 82 | 49 |
| Placebo | n | 42 | 34 | 36 | 18 | 22 | 14 | 42 | 22 |
| | % | 81 | 65 | 69 | 35 | 42 | 27 | 81 | 42 |

TABLE 3A
Duration of Follow-Up (Per-Protocol)

| | | Number of Subjects with OM Assessments | | | | | | | | | |
|---|---|---|---|---|---|---|---|---|---|---|---|
| Treatment Group | | V2 | V2 mid | V3 | V3 mid | V4 | V4 mid | V5 | V5 mid | V6 | V6 mid |
| Composition | n | 45 | 43 | 44 | 45 | 46 | 43 | 46 | 38 | 46 | 37 |
| 1 | % | 98 | 93 | 96 | 98 | 100 | 93 | 100 | 83 | 100 | 80 |
| Placebo | n | 46 | 41 | 47 | 44 | 45 | 41 | 42 | 39 | 41 | 38 |
| | % | 96 | 85 | 98 | 92 | 94 | 85 | 88 | 81 | 85 | 79 |

TABLE 3B
Duration of Follow-Up (Per-Protocol)

| | | Number of Subjects with OM Assessments | | | | | | | |
|---|---|---|---|---|---|---|---|---|---|
| Treatment Group | | V7 | V7 mid | V8 | V8 mid | V9 | V10 | V11 | V12 |
| Composition | n | 41 | 38 | 40 | 19 | 21 | 17 | 38 | 28 |
| 1 | % | 89 | 83 | 87 | 41 | 46 | 37 | 83 | 61 |
| Placebo | n | 42 | 34 | 36 | 18 | 22 | 14 | 38 | 22 |
| | % | 88 | 71 | 75 | 38 | 46 | 29 | 79 | 46 |

Table 4 summarizes the incidence of severe OM (WHO grades 3 or 4) using a few different methods.

TABLE 4
Maximum Observed WHO Score; subjects with no
post-baseline OM scores assigned Severe OM

| | | |
|---|---|---|
| Parameter/Subgroup | Composition 1 | Placebo |

| | | | | |
|---|---|---|---|---|
| Incidence of Severe OM (ITT) | 29/57 | (50.9%) | 31/52 | (59.6%) |
| Baseline MTS Pain = No and | 8/17 | (47.1%) | 13/23 | (56.5%) |
| Cisplatin = Q1 W | | | | |
| Baseline MTS Pain = No and | 5/11 | (45.5%) | 6/12 | (50.0%) |
| Cisplatin = Q3 W | | | | |
| Baseline MTS Pain = Yes and | 8/16 | (50.0%) | 10/14 | (71.4%) |
| Cisplatin = Q1 W | | | | |
| Baseline MTS Pain = Yes and | 8/12 | (66.7%) | 2/3 | (66.7%) |
| Cisplatin = Q3 W | | | | |

These results suggest that composition 1 lowered the incidence of severe OM among patients with baseline pain. The results also suggest that the effect is pronounced in patients who received the Q1W cisplatin regimen. Baseline pain is defined by MTS scores of 1 or greater versus 0=no pain within past 24 hours. MTS scores were assessed using a validated questionnaire.

It is suggested that Composition 1 works best when used to prevent OM during low intensity chemotherapy, as shown by the decrease in maximum observed score in the Q1W Composition 1 treatment group, relative to the placebo group, especially in those having pain within the past 24 hours, when compared to the high Q3W group.

AUC curves: For each subject, the MTS scores at each time point were used to calculate area-under-the-curve (AUC) using the trapezoidal rule. Table 5 (ITT) and Table 6 (Per Protocol) summarize the mean and median AUC values in each treatment group by visit and overall. These self-rated pain scores show a general trend favoring the active treatment group.

TABLE 5
AUC for MTS Scores (ITT)

| | | AUC for MTS Change Scores by Visit and Overall | | | | | | | | | |
|---|---|---|---|---|---|---|---|---|---|---|---|
| Treatment Group | | V3 | V4 | V5 | V6 | V7 | V8 | V9 | V10 | V11 | Overall |
| Comp. 1 | n | 46 | 46 | 43 | 37 | 34 | 31 | 24 | 16 | 26 | 52 |
| | Mean | 3.2 | 6.1 | 5.8 | 6.2 | 8.0 | 8.1 | 10.0 | 7.9 | 0.7 | 40.7 |
| | Median | 1.8 | 5.8 | 5.5 | 5.5 | 6.3 | 8.0 | 7.0 | 4.8 | 0.0 | 33.0 |
| Placebo | n | 43 | 43 | 40 | 38 | 35 | 28 | 23 | 17 | 23 | 48 |
| | Mean | 3.9 | 7.4 | 7.6 | 7.2 | 9.9 | 6.6 | 11.3 | 6.0 | 1.7 | 48.9 |
| | Median | 2.0 | 6.5 | 7.3 | 6.0 | 8.5 | 6.8 | 10.5 | 6.0 | 0.0 | 35.0 |

TABLE 6
AUC for MTS Scores (Per Protocol)

| | | AUC for MTS Change Scores by Visit and Overall | | | | | | | | | |
|---|---|---|---|---|---|---|---|---|---|---|---|
| Treatment Group | | V3 | V4 | V5 | V6 | V7 | V8 | V9 | V10 | V11 | Overall |
| Comp. 1 | n | 40 | 41 | 41 | 36 | 34 | 31 | 24 | 16 | 23 | 45 |
| | Mean | 3.4 | 5.9 | 5.9 | 6.4 | 8.0 | 8.1 | 10.0 | 7.9 | 0.8 | 45.4 |
| | Median | 2.5 | 6.0 | 5.5 | 5.5 | 6.3 | 8.0 | 7.0 | 4.8 | 0.0 | 40.5 |
| Placebo | n | 41 | 42 | 40 | 38 | 35 | 28 | 23 | 17 | 22 | 45 |
| | Mean | 3.7 | 7.5 | 7.6 | 7.2 | 9.9 | 6.6 | 11.3 | 6.0 | 1.8 | 51.7 |
| | Median | 2.0 | 7.0 | 7.3 | 6.0 | 8.5 | 6.8 | 10.5 | 6.0 | 0.0 | 45.5 |

Composition 1 appeared to: reduce the incidence of severe OM in patients who started the once-weekly cisplatin regimen with pre-existing mouth pain, and (2) lower the overall level of patient-reported mouth and throat soreness (AUC), as measured recorded daily during CRT.

It should be noted that the randomized groups showed an imbalance in the level of baseline pain: On average, the baseline MTS score was higher in the Composition 1 group than in the placebo group for both the ITT (0.904 vs. 0.563) and Per Protocol (0.956 vs. 0.600) populations. Likewise, the active treatment group had a higher percentage of patients with pain at baseline (52% vs. 33% for ITT; 53% vs. 36% for Per Protocol). However, the use of “change from baseline” for AUC calculations helps to adjust for this imbalance.

These results indicate that Composition 1 is a safe for OM, in patients undergoing cisplatin chemotherapy on a weekly basis. Similarly, it has been shown that Composition 1 is effective in reducing pain in OM patients, especially in those suffering from pain upon initiation of the treatment.

Brilacidin-OM is a drug in development by Innovation Pharmaceuticals for oral mucositis, containing brilacidin, a defensin mimetic. When tested in Sever Oral Mucositis (SOM) in Head and Neck Cancer (HNC), Brilacidin-OM was more effective in decreasing the incidence of SOM in HNC patients receiving the more aggressive chemotherapy regimen—cisplatin administered in a higher concentration (80-100 mg/m²), every 21 days—as compared to lower concentrations of cisplatin (30-40 mg/m²) administered weekly. (http://www.ipharmine.com/press-release/2018/4/9/innovation-pharmaceuticals-phase-2-oral-mucositis-trial-additional-data-show-brilacidin-om-demonstrated-a-significant-reduction-in-the-incidence-of-severe-oral- mucositis) Surprisingly, Composition 1 has shown higher effect in decreasing the incidence of OM in HNC patients receiving the less aggressive chemotherapy regimen.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method for prevention and/or treatment of oral mucositis in a patient in need thereof, wherein the patient is treated with a once-weekly regimen of 30-40 mg/m² of cisplatin, comprising, administering to the patient, a composition comprising extracts from the plant species Sambucus nigra, Echinacea purpurea, and Centella asiatica.

2. The method according to claim 1 wherein the composition is in the form of an oral rinse.

3. The method according to claim 2 wherein the herbal extracts are extracts produced by a first hydroalcoholic extraction from plant matter, followed by a second hydroalcoholic extraction of the first extract.

4. The method according to claim 1 wherein the ratio of Sambucus nigra:Echinacea purpurea:Centella asiatica extracts in the combination is 7:1:2.

5. The method according to claim 1 wherein the patient is undergoing radiotherapy.

6. The method according to claim 5 wherein the cumulative dose of radiation is up to 65 Gy.

7. (canceled)

8. The method according to claim 1 wherein the herbal extract is present in an amount of 1% of the composition.

9. The method according to claim 2 wherein the oral rinse is administered in an amount of 15 ml per administration.

10. The method according to claim 9 wherein the oral rinse is administered between 1 and 5 times per day.

11. The method according to claim 10 wherein the oral rinse is administered 3 times per day.

12. (canceled)

13. The method according to claim 1 wherein the patient suffers from head/neck cancer.

14. (canceled)

15. A method for treatment of pain associated with oral mucositis in a patient in need thereof, wherein the patient is treated with cisplatin, comprising, administering to the patient, a composition comprising extracts from the plant species Sambucus nigra, Echinacea purpurea, and Centella asiatica.

16. The method according to claim 15 wherein the composition is in the form of an oral rinse.

17. The method, according to claim 15, wherein the patient is treated with a once-weekly regimen of 30-40 mg/m² of cisplatin.

18. The method according to claim 15 wherein the herbal extracts are extracts produced by a first hydroalcoholic extraction from plant matter, followed by a second hydroalcoholic extraction of the first extract.

19. The method according to claim 15 wherein the ratio of Sambucus nigra:Echinacea purpurea:Centella asiatica extracts in the combination is 7:1:2.

20. The method according to claim 15 wherein the patient is undergoing radiotherapy.

21. The method according to claim 20 wherein the cumulative dose of radiation is up to 65 Gy.

22-27. (canceled)

28. The method according to claim 15 wherein the patient suffers from pain associated with oral mucositis as determined by an MTS score of 1 or higher during the 24 hours preceding administration of the cisplatin.

29. (canceled)

30. A method for treatment of pain associated with oral surgery in a patient in need thereof, wherein the patient is treated with cisplatin, comprising, administering to the patient, a composition comprising extracts from the plant species Sambucus nigra, Echinacea purpurea, and Centella asiatica.
